SEQUENCE LISTING (15 sequences)

SEQ ID NO: 1 - protein, 441 aa, Desmodus rotundus
AYGVACKDEITQMTYRRQESWLRPEVRSKRVEHCQCDRGQARCHTVPVNSCSEPRCFNGGTCWQAVYFSDFVCQCPAGYTGKRCEVDTRATCYEGQGVTYRGTWSTAESRVECINWNSSLLTRRTYNGRMPDAFNLGLGNHNYCRNPNGAPKPWCYVIKAGKFTSESCSVPVCSKATCGLRKYKEPQLHSTGGLFTDITSHPWQAAIFAQNRRSSGERFLCGGILISSCWVLTAAHCFQESYLPDQLKVVLGRTYRVKPGEEEQTFKVKKYIVHKEFDDDTYNNDIALLQLKSDSPQCAQESDSVRAICLPEANLQLPDWTECELSGYGKHKSSSPFYSEQLKEGHVRLYPSSRCAPKFLFNKTVTNNMLCAGDTRSGEIYPNVHDACQGDSGGPLVCMNDNHMTLLGIISWGVGCGEKDVPGVYTKVTNYLGWIRDNMHL

SEQ ID NO: 2 - DNA, 1326 bp, Desmodus rotundus
gcatatggtgtggcctgcaaagacgaaataacccagatgacataccggcgacaagagtcgtggctgcgccccgaggtcagaagcaagcgggtggaacactgccagtgcgatagaggccaggcccggtgccacaccgtgcccgtcaacagttgcagtgaaccaaggtgcttcaatggggggacatgctggcaggctgtatatttctcagactttgtctgtcagtgccctgcaggatatacggggaaacggtgtgaagtagatacccgtgccacctgctatgagggccagggtgtcacctacaggggcacatggagcacagcagaaagtagggttgagtgtatcaactggaacagcagccttctgacccggaggacctacaatgggcggatgccagatgccttcaacctgggccttgggaatcacaattactgcagaaacccaaatggagccccaaaaccttggtgctatgtcatcaaggcagggaagttcacctcggagtcctgtagcgtgcctgtctgctccaaggccacctgtggcctgagaaagtacaaggagccacagcttcacagtacaggaggactcttcacagacatcacctctcatccatggcaggctgccatctttgcccagaacagaaggtcatcaggagaaaggttcttgtgtggggggatattgatcagttcctgctgggtcctgactgctgcccactgcttccaggagagctatcttcctgaccagcttaaggtggttttgggcagaacataccgggtgaaacctggagaggaagagcagacatttaaagtcaaaaaatacatcgtccataaggaatttgatgacgacacttacaacaatgacattgcactgctgcagctgaaatcggactcaccacagtgtgcccaagagagtgacagtgtccgcgccatctgtctcccggaagccaacctgcagctgcccgactggacagaatgtgagctgtctggctacggcaagcataagtcatcttctcctttctattctgagcagctgaaggaagggcatgtcaggctgtacccctccagccgctgcgcacccaagtttctgtttaacaaaaccgtcacaaacaacatgctgtgtgctggagacacgcggagcggagagatctatccaaatgtgcacgatgcctgccagggtgactcaggaggccccttggtgtgtatgaatgacaaccacatgactttgcttggcatcatcagttggggtgttggctgtggggagaaagacgttccaggtgtatacaccaaggttactaattacctaggctggattcgagacaacatgcacctgtaa

SEQ ID NO: 3 - protein, 562 aa, Homo sapiens [sequence omitted]

SEQ ID NO: 4 - DNA, 1689 bp, Homo sapiens
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcatatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagatgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtga

SEQ ID NO: 5 - DNA, 1689 bp, Artificial
  Human t-PA gene codon optimized for expression in tobacco seeds
atggatgctatgaaaagaggattgtgttgcgttttgcttttgtgtggagccgtttttgtctctccatcccaagagattcatgctagattcagaagaggtgctagatcctaccaggtcatttgtagagatgaaaagacacaaatgatctatcaacagcaccagtcatggcttagacctgttttgagaagtaacagagtcgagtactgttggtgcaattctggtagagcccaatgtcatagtgttccagtcaaatcatgtagtgaacctagatgctttaacggtggaacttgtcaacaggctttgtacttctcttacttcgtttgtcaatgcccagagggattcgctggtaaatgttgcgaaattgacaccagagctacttgttacgaagatcagggaatctcatatagaggtacatggtctaccgctgagtccggagccgaatgtactaactggaattcttccgctttggcccaaaaaccatactctggtagaagacctgatgctattagacttggtttgggaaaccacaattattgcagaaatccagacagagattctaagccttggtgttacgtttttaaggccggaaaatattcaagtgaattctgttccacccctgcatgctcagagggtaacagtgattgttactttggtaacggttctgcttatagaggaacccattccttgactgagtcaggtgccagttgtcttccatggaactcaatgattttgatcggaaaagtttacactgcacaaaatcctagtgcacaggctcttggtttgggaaagcataactactgtagaaatccagacggagatgccaaaccttggtgtcacgttcttaagaacagaagattgacatgggaatactgtgacgtcccatcttgttccacctgcggtttgagacaatactcacaacctcagtttagaattaaaggtggattgttcgctgatatcgcctctcatccatggcaggctgccatttttgctaagcacagaagatcccctggagagagattcctttgtggtggaattttgatctcttcctgctggattttgtccgcagctcactgttttcaagaaagattcccacctcatcaccttacagttatcttgggaagaacctacagagttgtcccaggtgaagaggaacagaagtttgaggttgaaaaatacattgtccataaggagttcgatgacgatacttatgacaatgatatcgcacttttgcaattgaagtctgattcaagtagatgtgctcaggaatcttccgttgtcagaactgtttgtttgccacctgctgaccttcaattgcctgattggacagagtgtgaactttctggttacggaaaacacgaagccttgtctccattttattccgagagacttaaggaagcacatgttagattgtatccttcaagtagatgtacatcccaacaccttttgaacagaactgtcacagacaatatgttgtgtgctggagataccagatcaggtggaccacaagccaacttgcatgacgcatgccagggagatagtggtggacctcttgtttgtttgaatgacggtagaatgactcttgtcggaattatctcttggggtttgggatgtggtcaaaaagatgttccaggtgtctacactaaggttacaaactatttggactggatcagagataacatgagaccatga

SEQ ID NO: 6 - protein, 527 aa, Homo sapiens
SYQVICRDEKTQMIYQQHQSWLRPVLRSNRVEYCWCNSGRAQCHSVPVKSCSEPRCFNGGTCQQALYFSYFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 7 - DNA, 1581 bp, Homo sapiens
tcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcatatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagatgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccg

SEQ ID NO: 8 - DNA, 57 bp, Artificial Sequence
  Synthetic nucleic acid sequence encoding LPH19-amino-acid leader peptide
atggagtgtaattggatacttccttttattctcagtgtgaccagtggagcttattct

SEQ ID NO: 9 - protein, 19 aa, Artificial Sequence
  Synthetic amino acid sequence of LPH19-amino-acid leader peptide
MECNWILPFILSVTSGAYS

SEQ ID NO: 10 - DNA, 1470 bp, Phaseolus vulgaris
gaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatggcactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttc

SEQ ID NO: 11 - DNA, 34 bp, Phaseolus vulgaris
atcatccatccatccagagtactactactctact

SEQ ID NO: 12 - DNA, 64 bp, Tobacco mosaic virus
gtatttttacaacaattaccaacaacaacaaacaacaaacaacattacaattactatttacaat

SEQ ID NO: 13 - protein, 537 aa, Artificial Sequence
  Synthetic chimeric t-PA protein
SYQVICRDEKTQMIYQQHQSWLRPVLRSNRVEYCWCNSGRAQCHSVPVKSCSEPRCFNGGTCQQALYFSYFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRPHHHHHHKDEL

SEQ ID NO: 14 - protein, 4 aa, Artificial Sequence
  Synthetic amino acid endoplasmic reticulum retention sequence
KDEL

SEQ ID NO: 15 - protein, 8 aa, Artificial Sequence
  Synthetic tagging sequence
DYKDDDDK

(19) United States
(12) Patent Application Publication
(10) Pub. No.: US 2016/0208274 A1
(43) Pub. Date: Jul. 21, 2016

(54) BLOOD CLOT-DISSOLVING PROTEINS PRODUCED IN SEEDS

(71) Applicant: NORTHEASTERN STATE UNIVERSITY, Tahlequah, OK (US)

(72) Inventor: Kevin Yueju Wang, Tulsa, OK (US)

(21) Appl. No.: 15/002,087

(22) Filed: Jan. 20, 2016

Related U.S. Application Data
(60) Provisional application No. 62/106,068, filed on Jan. 21, 2015.

(51) Int. Cl. C12N 15/82; C12N 9/72

(52) U.S. Cl. CPC C12N 15/8251; C12N 9/6459; C12Y 304/21068

(57) ABSTRACT

Transgenic plants in which blood-clot dissolving proteins are produced in seeds of the plants are provided. Expression of the proteins is driven by a seed specific or selective promoter. Exemplary blood-clot dissolving proteins produced in this manner include recombinant Desmodus rotundus salivary plasminogen activator α1 (DSPAα1) and recombinant human tissue plasminogen activator (t-PA). Recombinant proteins isolated from seeds dissolved blood clots.

### CROSS REFERENCE TO RELATED APPLICATIONS

This application claims the priority of U.S. Provisional Patent Application No. 62/106,068 titled “SEED-DERIVED BLOOD CLOT-DISSOLVING PROTEINS,” filed Jan. 21, 2015, the contents of which are hereby incorporated by reference.

### STATEMENT REGARDING FEDERALLY SPONSORED RESEARCH OR DEVELOPMENT

This invention was made with government support under grant number R03NS095246 (PI) awarded by the National Institute of Health-National Institute of Neurological Disorders and Stroke, grant number P20GM103447 (PI) awarded by the National Institute of Health-INBRE Research Project Investigator Award, and grant number P20RR016478 (PI) awarded by the National Institute of Health-INBRE Junior Investigator Award. The government has certain rights in the invention.

### FIELD OF THE INVENTION

The invention relates to the use of transgenic plant seeds to produce therapeutic proteins. In particular, the invention relates to transgenic tobacco plant lines used for production of Desmodus rotundus salivary plasminogen activator, DSPAα1 and tissue plasminogen activator (t-PA) in tobacco seed using a seed specific promoter.

### BACKGROUND OF THE INVENTION

Currently, recombinant tissue-type plasminogen activator (rt-PA) is the only FDA-approved drug for the treatment of acute, ischemic strokes. It is a serine protease present in all vertebrate species that have been thus far investigated (Lijnen and Collen 1987). This enzyme catalyzes the conversion of plasminogen to active plasmin, which can degrade many blood plasma proteins; most notably fibrin clots. t-PA is the primary enzyme responsible for the breakdown of blood clots (Suzuki et al., 2009). Although t-PA has some limitations and side effects, such as a short treatment window (3-4.5 h after a stroke occurs), increased bleeding, and risk of brain injury (Adams et al., 2007; Hacke et al., 2008; Tsirka et al., 1995); it is still the most commonly used drug, worldwide, for dissolving major blood clots before they induce tissue death as a result of oxygen deprivation.

Scientists have identified plasminogen activators from vampire bat (Desmodus rotundus) saliva (D. rotundus salivary plasminogen activator, DSPA) (Kratzschmar et al., 1991, 1992). DSPAα1 and DSPAα2 have significantly greater specificity for fibrin than tissue-plasminogen activator (Bringmann et al., 1995) which allows these enzymes to dissolve a clot locally without affecting the entire blood coagulation system. Studies have shown that DSPAα1 is safe in patients with acute ischemic stroke even when given up to 9 hours after stroke onset. DSPAs do not display the neurotoxic effects seen with tissue plasminogen activator (t-PA, sold as alteplase, reteplase, and tenecteplase). DSPAs therefore hold great promise as new plasminogen activators for stroke patients (Dafer and Biller 2007; Furlan et al., 2006; Grandjean et al., 2004; Lijnen and Collen 2000).

Common microbial hosts such as E. coli can produce high yields of recombinant protein, but lack the requisite machinery for post-translational modification (Lilie et al., 1998; Ma et al., 2005). Animal cell systems can be used to produce biologically active human pharmaceutical protein. However, they are very costly. Over the last decade, plants have emerged as convenient and economical alternative expression systems (Ma et al., 2005). Plant molecular farming (PMF) is expected to challenge established production technologies that use bacteria, yeast or cultured mammalian cells (Ma et al., 2005; Peterson and Artzen 2004).

Plant expression systems have major advantages over other prokaryotic and eukaryotic expression systems in terms of speed, cost, and safety. The yield of protein per wet tissue weight can be many times larger than that obtained using microbial or animal-cell-based systems. Most importantly, plant systems have the potential to be far less expensive platforms for the production of medicinal proteins (Bock and Warzecha 2010; Spök et al., 2008). Currently, most pharmaceutical proteins are synthesized in aqueous leafy crops for biomass. However, proteins synthesized in this manner are subject to rapid proteolytic degradation after harvest (Dorana 2006).

It would be of great benefit to have available alternative methods and systems for stably producing recombinant proteins in commercially viable quantities in a cost effective manner.

### SUMMARY OF THE INVENTION

The present disclosure describes methods of producing recombinant blood clot dissolving proteins by targeting the production of the proteins to the seeds of plants. The production of proteins in this manner avoids proteolytic and other degradation that is typically associated with protein production in non-seed portions of plants. In addition, the yield of protein generally exceeds that which is produced using other systems such as mammalian and bacterial systems, and at a lower cost. Thus, using the methodology disclosed here, recombinant proteins are made in abundance in a cost effective manner. The production of proteins in seeds also advantageously allows for long-term stability of unpurified protein, e.g. during storage of seeds at room temperature, without detectable loss of protein activity after purification. In an exemplary aspect, the recombinant proteins targeted for production in plant seeds are the blood clot-dissolving proteins DSPA (e.g. DSPA-α1) and tissue plasminogen activator (tPA).

The invention provides transgenic seeds comprising a protein that dissolves blood clots. In some aspects, the transgenic seed of claim 1, wherein the transgenic seed is from a plant type selected from the group consisting of tobacco, rice, maize and soybean. In some aspects, the protein that dissolves blood clots is Desmodus rotundus salivary plasminogen activator (DSPA) or human tissue plasminogen activator (t-PA). In other aspects, the DSPA is or includes an amino acid sequence as set forth in SEQ ID NO: 1 and the t-PA is or includes an amino acid sequence as set forth in SEQ ID NO: 6.

The invention further provides transgenic plants or progeny thereof, comprising a nucleic acid sequence which includes a nucleotide sequence encoding a protein that dissolves blood clots operably linked to a seed specific or selective promoter. In some aspects, the transgenic plant or progeny thereof is a type of plant selected from the group consisting of tobacco, rice, maize and soybean. In some aspects, the protein that dissolves blood clots is Desmodus rotundus salivary plasminogen activator (DSPA) or human tissue plasminogen activator (t-PA). In additional aspedts, the seed specific or selective promoter is a phaseolin promoter or a napin promoter.

In addition, the invention provides methods of making a recombinant protein that dissolves blood clots. The methods comprise steps of i) genetically engineering a plant cell or a plant explant to contain and express a nucleotide sequence encoding a protein that dissolves blood clots operably linked to a seed specific or selective promoter; ii) cultivating the plant cells or plant explant so as to produce a transgenic plant, iii) cultivating the transgenic plant so as to produce seeds comprising the protein that dissolves blood clots; iv) harvesting the seeds; and iv) isolating the protein that dissolves blood clots from the seeds.

In further aspects, the invention provides vectors comprising a nucleotide sequence encoding a protein that dissolves blood clots operably linked to a seed specific or selective promoter. In some aspects, the nucleic acid sequence that is present in the vector includes a nucleotide sequence as set forth in SEQ ID NO 2, SEQ ID NO: 4 or SEQ ID NO: 5. In further aspects, the nucleic acid sequence encoding a protein encodes an amino acid sequence which is or includes an amino acid sequence as set forth in SEQ ID NO: 1, or an amino acid sequence which is or includes an amino acid sequence as set forth in SEQ ID NO: 6.

Further aspects of the invention provide nucleotide sequences which include a nucleotide sequence encoding a blood clot-dissolving protein operably linked to a seed specific or selective promoter. In some aspects, the encoded blood clot-dissolving protein is DSPA. In certain aspects, the DSPA that is encoded is or includes an amino acid sequence as set forth in SEQ ID NO: 1. In further aspects, the DSPA is encoded by a nucleotide sequence that is or includes a nucleotide sequence as set forth in SEQ ID NO: 2. In further aspects, the blood clot-dissolving protein is t-PA. In certain aspects, the t-PA is or includes an amino acid sequence as set forth in SEQ ID NO: 6. In additional aspects, the t-PA is encoded by a nucleotide sequence that is or includes a nucleotide sequence as set forth in SEQ ID NO: 4 or SEQ ID NO: 5. In some aspects, the seed specific or selective promoter is or includes a nucleotide sequence as set forth in SEQ ID NO: 10.

Further aspects of the invention provide recombinant proteins which include an amino acid sequence as set forth in SEQ ID NO: 1 or SEQ ID NO: 6.

### BRIEF DESCRIPTION OF THE DRAWINGS

FIG. 1A. Plant expression vectors for A, DSPA and B, t-PA. The seed-specific phaseolin (phas) promoter was used to drive expression in tobacco. Each protein was targeted to the ER using KDEL peptides. The following abbreviations are used: phas: Bean seed-specific phaseolin (phas) promoter; nosT: polyadenylation signal of nopaline synthase; RB/LB: Plant T-DNA right/left border; NPTII: Neomycin Phosphotransferase (kanamycin resistance) plant selectable marker; Ω: 5′-untranslated sequence of tobacco mosaic virus RNA (Ω enhancer); LPH: plant optimized murine mAb24 heavy chain. 6×His was used for protein purification purposes.

FIG. 1B. Plant expression vectors for A, DSPA and B, t-PA. The seed-specific phaseolin (phas) promoter was used to drive expression in tobacco. Each protein was targeted to the ER using KDEL peptides. The following abbreviations are used: phas: Bean seed-specific phaseolin (phas) promoter; nosT: polyadenylation signal of nopaline synthase; RB/LB: Plant T-DNA right/left border; NPTII: Neomycin Phosphotransferase (kanamycin resistance) plant selectable marker; plant optimized murine mAb24 heavy chain. 6×His was used for protein purification purposes.

FIG. 2A. Fibrin plate assays. Fibrin plate screening of tobacco seed-derived t-PA: CK=10 units of commercial human t-PA; 1, 2 and 3: 50 uL (each) of eluant from t-PA transgenic T1 tobacco seeds.

FIG. 2B. Fibrin plate assays. Fibrin plate assay of DSPAα1: t-PA=10 units of commercial human t-PA; LPH-DSPAα1=50 μL of eluant from DSPAα1 transgenic tobacco T3 seeds; wt: 50 μL of eluant from non-transgenic tobacco seeds.

FIG. 3A. Blood-clot lysis assay. Blood clots in phosphate buffered saline, no additives.

FIG. 3B. Blood-clot lysis assay. t-PA: 10 units of commercial human t-PA; LPH-DSPAα1:50 μL of elutant from T3 seeds; wt: 50 μL of elutant from non-transgenic seeds; PBS: 50 μL of phosphate buffered saline.

FIG. 4A. DSPPα1. Amino acid sequence of mature DSPPα1 (SEQ ID NO: 1).

FIG. 4B. DSPPα1. DNA sequence encoding mature DSPPα1 (SEQ ID NO: 2).

FIG. 5A. t-PA. Amino acid sequence of full-length t-PA (before posttranslational processing) (SEQ ID NO: 3).

FIG. 5B. t-PA. DNA sequence encoding full-length t-PA (SEQ ID NO: 4).

FIG. 5C. t-PA. Codon optimized DNA sequence encoding full-length t-PA (SEQ ID NO: 5).

FIG. 5D. t-PA. Amino acid sequence of mature t-PA (after posttranslational processing) (SEQ ID NO: 6).

FIG. 5E. t-PA. DNA sequence encoding mature t-PA (SEQ ID NO: 7).

FIG. 6A. Nucleic acid sequence encoding LPH:19-amino-acid leader peptide from the heavy chain of murine monoclonal antibody (SEQ ID NO: 8).

FIG. 6B. Amino acid sequence of LPH:19-amino-acid leader peptide (SEQ ID NO: 9).

FIG. 6C. Nucleic acid sequence of the seed-specific phaseolin (phas) promoter (SEQ ID NO: 10).

FIG. 6D. Phas protein 5′-UTR DNA sequence (SEQ ID NO: 11).

FIG. 6E. The 5′-untranslated sequence of tobacco mosaic virus RNA (1 enhancer) (SEQ ID NO: 12).

FIG. 7. Predicted amino acid sequence of the entire protein as translated (prior to any post-translational modification) for t-PA (“t-PA-6His-KEDL”; SEQ ID NO: 13).

### DETAILED DESCRIPTION OF THE PREFERRED EMBODIMENTS

The present invention provides recombinant proteins which dissolve, degrade or break down blood clots and which are targeted so as to be produced in plant seeds. As described herein, recombinant constructs from which the proteins are produced contain seed specific promoters which preferentially target production of the proteins in plant seeds. Exemplary proteins of this type include but are not limited to DSPAα1 and t-PA.

The proteins DSPAα1 and t-PA both have the ability to dissolve blood clots and as such represent valuable tools in the treatment of diseases and conditions involving unwanted clots (thrombi and emboli). As described herein, large quantities of these proteins can be produced in stable form and in a cost effective manner when production is targeted to plant seeds. Proteins that are produced as described herein are used, for example, in the treatment of various diseases and conditions involving unwanted blood clots.

The following definitions are used throughout:

tPA (or PLAT) is a serine protease (EC 3.4.21.68) found on endothelial cells that line blood vessels. tPA catalyzes the conversion of plasminogen to plasmin, the major enzyme responsible for clot breakdown. tPA is used to treat e.g. embolic and thrombotic stroke. As used herein, “t-PA” can refer to human other other, usually mammalian, forms of the protein and the gene encoding the protein.

Desmodus rotundus (vampire bat) salivary plasminogen activator al (DSPAα1 or desmoteplase (INN)) is a plasminogen activator with high fibrin specificity. This high fibrin specificity makes DSPAα1α promising candidate for the treatment of acute ischemic stroke. In particular, DSPAα1 can be used as a replacement for, and alternative to or in conjunction with t-PA, which can cause neurotoxic effects and unwanted bleeding, e.g. intracranial bleeding, and is recommended for use only within the first few hours after a stroke.

A thrombus, or blood clot, is the final product of the blood coagulation step in hemostasis. There are two components to a thrombus: aggregated platelets that form a platelet plug, and a mesh of cross-linked fibrin protein. A thrombus is a healthy response to injury intended to prevent bleeding, but can be harmful in thrombosis, when clots obstruct blood flow through healthy blood vessels.

Thrombosis is the formation of a blood clot inside a blood vessel, obstructing the flow of blood through the circulatory system. When a blood vessel is injured, the body uses platelets (thrombocytes) and fibrin to form a blood clot to prevent blood loss. However, even when a blood vessel is not injured, blood clots may form in the body under certain conditions, causing extensive damage, due to oxygen deprivation, of the area which is otherwise serviced by the blood vessel, e.g. peripheral arterial thrombi and thrombi in the proximal deep veins of the leg. A clot that breaks free (embolism) and travels through the circulatory system can be extremely dangerous and cause an embolism if it becomes “stuck” in a blood vessel.

Embolism: obstruction of a blood vessel such as an artery, typically by a clot of blood that has broken free and traveled from the location in which it was originally formed. Embolisms can occur at many locations and can cause extremely serious conditions e.g. an arterial embolism in the brain (cerebral embolism) causes stroke, which can be fatal. In a pulmonary embolism, blood flow is blocked at a pulmonary artery. When the main pulmonary artery is blocked, the embolism can quickly become fatal. More than 90% of cases of pulmonary emboli are complications of deep vein thrombosis (DVT) a blood clot that has formed in one or more of the deep veins in your body, usually in your legs.

Thromboembolism is the term used to describe the combination of thrombosis and its main complication, embolism.

Stroke: rapid decline of brain function due to a disturbance in the supply of blood to the brain, e.g. due to ischemia, thrombus, embolus or hemorrhage.

“Seed specific promoter”: drives production of a protein only in seeds; “Seed selective promoter”: drives most production of a protein in seeds, e.g. at least about 50, 60, 70, 80 or 90% or more of the protein is produced in seeds.

A protein of interest as described herein is a protein that dissolves, degrades, or breaks down blood clots, or which causes the dissolution, degradation or breakdown of blood clots, either directly or indirectly. The gene encoding the protein is transcribed and translated within the seeds of a plant that has been genetically modified to contain a vector that comprises at least one nucleic acid gene sequence encoding the protein and a plant seed specific (or selective) promoter. The vector is designed (i.e. the elements of the vector are arranged) so that the sequence encoding the protein and the sequence of the specific/selective promoter are operably linked, i.e. expression of the protein is driven by the seed specific/selective promoter, resulting in expression of the protein either exclusively or selectively in seeds.

Exemplary seed specific/selective promoters that may be used in the practice of the invention include but are not limited to e.g. Arabodopsis promoters Pro-at3g03230 (expressed in chalazal endosperm), Pro-at4g27530:GUS (expressed in chalazal endosperm and embryo), Pro-at4g31830 (expressed in radicle and procambium), Pro-at5g10120 and Pro-at5g16460 (expressed in embryo), Pro-at5g53100:GUS (expressed in endosperm), and Pro-at5g54000 (expressed in embryo and inner integument), DIRIGENT PROTEIN1 (DP1) gene promoter (seed coat specific expression); fragment BCSP666 of soybean promoter region of the β-conglycinin α-subunit gene; the seed specific gluteline 1 (Gt-1) promoter from rice disclosed in U.S. Pat. No. 7,192,774; the globulin-1 (Gb-1) promoter from rice; seed specific promoters described in US patent publication 20120036595 and in issued U.S. Pat. Nos. 5,623,067, 5,767,363, 7,371,928 and 8,404,926; Napin promoter from B. napus and B. campestris described in EP-A.2-0255378 and EP-A-0255377; Flax seed specific promoters described in US patent publication U.S. Pat. No. 7,642,346 B2. In preferred embodiments of the present invention the seed specific promoter used is a legumin-like seed storage protein promoter or a 2S storage protein promoter.

The “seed specific promoter” may be specific for gene expression in the entire seed or in one or more parts or types of cells of a seed. For example, the promoter may be specific/selective for gene expression in the seed coat, embryo, endosperm, tegmen, testa, raphe, integument, in palisade cells, in the fringe layer, etc. It may be a transcriptional initiation region and ribosome binding site from a gene expressed in a seed embryo or a seed coat cell or from a gene encoding a seed storage protein. It may be a sequence from a gene that encodes a product preferentially expressed in a plant seed cell as compared to other plant cells, as described, for example, in U.S. Pat. Nos. 5,608,152, 5,420,034, and EP 255378 B2.

Vectors which may be used to carry sequences encoding a protein of interest and a seed specific/selective promoter as described herein are typically plasmids that have been specifically designed to facilitate the generation of transgenic plants. In some aspects, they are binary vectors having the ability to replicate in both E. coli and e.g. in Agrobacterium tumefaciens, the bacterium that is frequently used to insert recombinant DNA into plants. As such, a suitable vector usually includes a transfer DNA (T-DNA) region for inserting the DNA into the agrobacteria prior to its introduction into cells of the plant. The vector may also comprise e.g. at least one selection gene (for example, for antibiotic resistance or another selectable trait), as well as various other genes and/or sequences required for replication of the plasmid, as known to those of skill in the art.

However, non-Agrobacterium vectors may also be employed, examples of which include but are not limited to: cauliflower mosaic virus vectors, cowpea mosaic virus vectors, bean pod mottle virus (BPMV) vectors, tobacco mosaic virus (TMV) vectors, potato virus X (PVX) vectors, Brome mosaic virus (BMV) vectors, bean yellow dwarf virus vectors, Gemini virus vectors, etc.

As indicated above, the gene sequences that are translated into proteins in plant seeds as described herein are, within a vector, operably linked to or positioned with respect to a seed specific/selective promoter that effects transcription of the gene sequence. In some aspects, at least one copy of the encoding gene is present, and multiple copies may be present in the vector. In addition, other sequences involved in protein production are generally also included. The additional sequences may be translated as part of the protein or may be regulatory sequences which are not translated. For example, the vector may comprise a suitable untranslated stop signal at the end of the coding sequence. Suitable stop sequences include but are not limited to: Nopaline synthase terminator (nos) and the 35S terminator derived from the Cauliflower Mosaic Virus (CaMV). Other non-translated sequences such as enhancer sequences, some transcription factors, and the like may also be present.

Exemplary translated sequences that may be present (and which are translated as port of the protein) include but are not limited to: various signal or targeting sequences which direct the movement of the translated protein within the plant, e.g. signal peptides including but not limited to plant optimized secretion signal mAb24 heavy chain (LPH, a leader peptide from the heavy chain of murine monoclonal antibody that enables transport of the protein to the apoplast); the PbTS leader peptide sequence (22 amino acids) that is derived from legu-minA2 of Pisum sativum (GenBank accession X17193) and targets native leguminA2 to protein bodies in pea seeds, the VTS⁴ leader sequence is derived from the strictosidine synthase gene of Catharanthus roseus (GenBank accession X61932) and comprises 28 amino acids (the C-terminal four serine residues from the native sequence were omitted since they would lead to incorrect cleavage as predicted by the CBS SignalP prediction server, see the website located at www.cbs.dtu.dk/services/SignalP-2.0/); etc.; sequences which direct or bias retention of the protein at a particular location and/or in a particular organelle of the plant, e.g. the amino acid sequence KDEL (SEQ ID NO: 14) for retention of the recombinant proteins in the endoplasmic reticulum (ER),), or the amino acid sequences KKMP distributes protein to the intermediate compartment and Golgi complex, etc.; sequences that facilitate protein purification e.g. histidine tags, Glutathione S-transferase (GST), the FLAG tag sequence DYKDDDDK (SEQ ID NO: 13), the Maltose-Binding Protein (MBP) tag, etc.

Generally, transformation is the introduction of DNA representing a cloned gene into a cell so that it expresses the protein encoded by the gene. Transformation processes include “indirect gene transfer”, where exogenous DNA is introduced by a biological vector, and “direct gene transfer”, where physical and chemical processes are responsible for DNA introduction. Transient expression represents the case in which vectors replicate within plant cells and the proteins are translated directly from the vectors. A stable transformation process demands the simultaneous occurrence of two independent biological events, which are: stable insertion of the transgene into the plant genome and regeneration of those cells where it occurred, producing a non-chimeric transgenic plant. While the foreign protein may be present throughout the plant, translation occurs solely or primarily in plant seeds if a seed-specific promoter used.

The invention also provides nucleotide sequences comprising sequences which encode a gene encoding a protein as described herein plus a promoter that is specific or selective for plant seeds. Other elements that are described above may also be present in the nucleotide sequence. The nucleotide sequence may be DNA, cDNA, RNA (e.g. mRNA) or hybrids of these. In some aspects, the nucleotide sequence is or includes a sequence as set forth in SEQ ID NO: 2 (which encodes DSPAα1 protein) and/or a sequence as set forth in SEQ ID NO: 4 (which encodes t-PA protein), or a sequence as set forth in SEQ ID NO: 5 (which encodes t-PA protein using a codon optimized sequence). In other aspects, the nucleotide sequences comprise one or both of SEQ ID NO: 2 and/or a sequence as set forth in SEQ ID NO: 4 and/a sequence as set forth in SEQ ID NO: 5 plus SEQ ID NO: 10, the nucleic acid sequence of the seed-specific phaseolin (phas) promoter. Also encompassed are sequences which encode the same proteins using different codons, and any nucleotide sequences which are at least about 90, 91, 92, 93, 94, 95, 96, 97, 98 or 99% homologous to the sequences.

The invention also encompasses proteins or polypeptides which comprise an amino acid sequences as set for in SEQ ID NO: 1 (DSPAα1) or SEQ ID NO: 3 (t-PA before posttranslational processing), or SEQ ID NO: 6 (t-PA after posttranslational processing), including proteins/polypeptides that are identical to those sequences, or proteins/polypeptides that comprise one of those sequences, e.g. fusion or chimeric proteins/polypeptides that comprise one or more of the proteins plus other sequences (e.g. other peptide/proteins sequences, signal sequences, various localization (e.g. retention) sequences, sequences which facilitate isolation of the polypeptide/protein, or adventitious sequences which are present due to vector-encoded sequences, or sequences which facilitate or simplify cloning of encoding sequences, etc. In other aspects, the invention encompasses proteins/polypeptides with or comprising amino acid sequences as set forth in SEQ ID NO: 12 (recombinant t-PA, as translated from the described in the Examples section below). Further, sequences with at least about 90, 91, 92, 93, 94, 95, 96, 97, 98, or 99% identity to any of these sequences are also encompassed, especially those comprising conservative amino acid substitutions. Those of skill in the art are familiar with the meaning of “conservative substitutions” e.g. wherein a positively charged amino acid is replaced by another positively charged amino acid, a negatively charged amino acid is replaced by another negatively charged amino acid, or a hydrophobic amino acid is replaced by another hydrophobic amino acid, etc. Any such substitutions are encompassed, so long as the resulting protein/polypeptide retains at least about 50, 55, 60, 65, 70, 75, 80, 85, 90, or 95% of the activity of the parent molecule, i.e. the conservative variant is a function or activity conservative variant.

In some aspects, the t-PA gene sequence that is used as the basis of transcription and translation of t-PA protein (a serine protease) in seeds as described herein is a human t-PA gene. However, this is not always the case. Other blood-clot dissolving seine proteases, such as lumbrokinase (LK) from earthworm, human Urokinase-type plasminogen activator (uPA), etc. may also be used.

Insertion of the vector into a host plant is generally accomplished using known techniques. For example, an Agrobacterium tumefaciens system may be used in which the bacteria are first transfected with a vector encoding the protein of interest (e.g. by electroporation) and then the A. tumefaciens bacteria are used to infect cells or explants or other tissue of a host plant of interest. However, other techniques for genetically modifying plants, examples of which include but are not limited to: the gene gun, microfibers, direct electroporation into plant cells, etc.

After cells or explants of a plant are genetically modified, they are cultivated by techniques known to those of skill in the art to produce adult plants and, for the purposes of the present invention, to produce seeds. For example, special soils and nutrients, specific growing conditions (e.g. photoperiods, sterile conditions, controlled moisture, etc.) may be employed in a green house or other controlled environment to produce adult plants that can then be transplanted and allowed to grow under conditions that permit seed formation.

Types of plants that produce seeds in which the proteins described herein may be made include but are not limited to: tobacco, maize, soybean, and rice, etc. Further, as used herein a genetically modified or transgenic “plant” includes all parts of the plant (e.g. stem, leaves, seeds, blossoms, reproductive organs, organelles, individual cells, explants, etc.), as well as progeny of the plant.

Recombinant, genetically engineered (modified) seeds are harvested from the plants by any suitable technique, including by hand and/or mechanically. Thereafter, the seeds may be stored indefinitely e.g. at room temperature until it is desired to isolate the protein of interest. Isolation of the protein is carried out e.g. by mechanically crushing, grinding or pulverizing the seeds and extracting the protein in a suitable solvent. Suitable solvents include aqueous solvents that are buffered, typically in a neutral pH range (e.g. from about 6.8 to about 8.8), such as extraction buffer comprised of 50 mM NaH₂PO₄, 300 mM NaCl, 10 mM 2-mercaptoethanol, 1% Polyvinylpyrrolidone, pH 8. Thereafter, the protein solution is treated as necessary to insure dissolution of the protein and readiness for further purification, e.g. by concentration, filtration, precipitation, etc. depending on the nature of the protein. If a “tag” (e.g. a His tag) is included in the protein sequences to facilitate isolation, an affinity column specific for the tag may be used to separate the protein from impurities. Otherwise, or in addition, other types of column chromatography may be used, or affinity columns based on a natural ligand of the protein, etc. Any suitable purification techniques may be used to achieve a desired level of purity of the protein.

Protein yields from the recombinant seeds described herein is generally in the range of from about 500 to about 1500 mg per kg of seed dry weight.

Purified protein is then further processed to produce compositions that are suitable for administration to a subject, such as a patient in need of blood clot dissolution, using techniques that are well known in the art. The compositions typically include one or more substantially purified proteins as described herein and a pharmacologically suitable carrier. The preparation of such compositions is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The liquids may be aqueous or oil-based suspensions or solutions. The active ingredients may be mixed with excipients which are pharmaceutically acceptable and compatible with the active ingredients, e.g. pharmaceutically acceptable salts. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of protein in the formulations may vary. However, in general, the amount in the formulations will be from about 1-99%. Still other suitable formulations for use in the present invention can be found, for example in Remington's Pharmaceutical Sciences, Philadelphia, Pa., 19th ed. (1995).

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The recombinant proteins described herein are used to prevent or treat a variety of conditions or diseases caused by an unwanted blood clot in a subject in need thereof. The proteins may dissolve or degrade clots directly, e.g. by attacking a component of the clot such as fibrin, which is enzymatically degraded by DSPAα1; or indirectly by promoting the synthesis of another protein in the clot destroying pathway, such as t-PA, which catalyzes the conversion of plasminogen to plasmin, the major enzyme responsible for clot breakdown. In some aspects, the blood clot is located in a blood vessel in tissue that, but for the presence of the blood clot, would be healthy. By “prevent” we mean that symptoms of the disease/condition have not yet occurred but the subject to whom the protein is administered is at risk of developing disease symptoms caused by an unwanted blood clot. A sufficient (efficacious) amount of the therapeutic active agent of interest, e.g. a recombinant protein as described herein, is administered to the subject to prevent or at least delay or lessen the degree of symptoms of the disease or condition. For example, the subject may have a clot (such as occurs in DVT) which is localized and has not broken free or traveled, but which is susceptible to doing so. In addition, a subject may be at risk of developing an unwanted blood clot, e.g. due to: impending or recent surgery such as heart or other surgery; or due to remaining stationary for a long period of time (e.g. during recuperation after an accident or during or after an illness), or after receipt of an artificial heart valve or stent, or a prosthesis, etc.

By “treat” we mean that the subject has already been diagnosed with a disease or condition caused or characterized by an unwanted blood clot. A sufficient (efficacious) amount of the therapeutic active agent of interest, e.g. a recombinant protein as described herein, is administered to the subject to alleviate, reverse or at least ameliorate symptoms of the disease or condition. Those of skill in the art will recognize that “prevention” and “treatment” may overlap, such as in the case of DVT: diagnosed DVT may be treated in order to dissolve the clot and thereby prevent the occurrence of a brain embolism and stroke. Exemplary conditions that may be prevented or treated using the proteins produced as described herein include but are not limited to: DVT, stroke, embolisms (e.g. arterial and venous embolisms, pulmonary embolism, brain embolism, retinal embolism, etc.), and the like.

### EXAMPLES

### Example 1

### Materials and Methods

Plant Expression Vector:

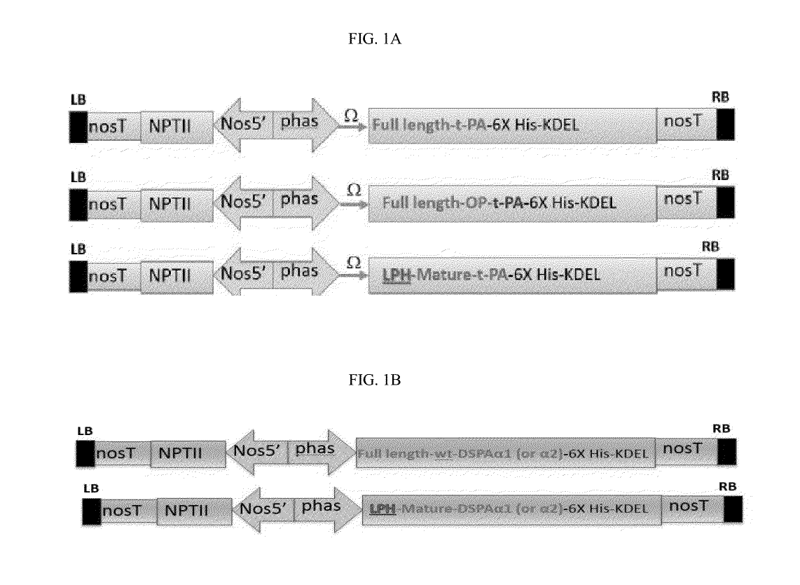

The coding sequences of the original full-length t-PA, codon-optimized full length t-PA, mature t-PA, and mature DSPAα1 and DSPAα2, were fused with a C-terminal 6×His tag and KDEL ((SEQ ID NO: 14); ER retention signal, Nuttall et al., 2002), respectively and synthesized by GenScript USA Inc. (Piscataway, N.J., USA). In order to increase the recombinant protein yields in plant cell compartments, we replaced t-PA, DSPAα1 and DSPAα2 signal peptides with the plant optimized murine mAb24 heavy chain (LPH:19-amino-acid leader peptide from the heavy chain of murine monoclonal antibody, 24) secretion signal. These targeting sequences enable transport of the t-PA and DSPA proteins to the apoplast. The LPH-t-PA, -DSPAα1 or -DSPAα2 gene sequences were flanked by C-terminal 6×His tags for protein purification, and KDEL (SEQ ID NO: 14) sequence for retention of recombinant proteins in the endoplasmic reticulum (ER). All gene fragments were synthesized by GenScript USA Inc. (Piscataway, N.J., USA) and inserted between a seed-specific phaseolin promoter (phas) and a nopaline synthase terminator (NosT) of the plant expression construct, pCambia2300-Phas1470-Nos (FIG. 1).

Plant Transformation

The plant expression vectors described above were introduced into ElectroMAX™ A. tumefaciens LBA4404 Cells (Life Technologies, USA) by an electroporation system (Eppendorf, Hamburg, Germany). The transformed reaction mixture was spread on LB agar plates with kanamycin (50 mg/L) and incubated at 28° C. After three days of incubation, a single colony was selected and, using a cotton swab, was spread out evenly on an LB agar plate with kanamycin (50 mg/L) and then incubated at 28° C. for two days. The culture was collected by a sterile scoop and re-suspended in MS liquid medium to obtain an OD₆₀₀ of approximately 0.4 to 0.6. Explants (0.5 cm×0.5 cm) were excised from 4- to 6-week-old sterile tobacco (Nicotiana tabacum SR1) seedlings and immersed in the Agrobacterium suspension described above for 30 to 40 min. The explants were then blotted on sterile filter paper and plated on a co-cultivation medium (MS, 6-BA 2.0 mg/L, acetosyringone 100 mg/L) in the dark for 4 days at 25° C. After co-culture, the explants were transferred onto selection medium (MS, 6-BA 2.0 mg/L, kanamycin 100 mg/L, cefotaxime 250 mg/L and carbenicillin 250 mg/L). Cultures were incubated at 25° C./23° C. (day/night temperature) with a 16-hr photoperiod. Explants were transferred to fresh selection medium every 2 weeks to generate shoots. Shoots were then transferred to a rooting medium (MS, sucrose 3.0%, kanamycin 100 mg/L) to obtain roots. Rooted plants were allowed to grow to 5-cm in Magenta® Plant Tissue boxes, and then transferred to soil.

Homozygous Transgenic Tobacco Line Development

Transgenic plant lines carrying the expression construct and having the highest level of tPA and DSPA protein expression in seeds were identified by fibrin plate assay. T1 seeds were obtained by screening plants subjected to transformation on media amended with kanamycin and then transferring the surviving plants to the soil for further growth and production of T1 seeds. T1 plants were grown in soil and self-fertilized to produce T2 seeds. T2 seeds were screened again on an agar medium amended with kanamycin, followed by transfer of the surviving plants to the soil where they were subjected to self-fertilization. Homologous T3 seeds were obtained from T2 plants using kanamycin selection medium.

His-Tagged Protein Extraction and Purification

Total soluble protein from dry mature seed (T1 t-PA and DSPAα2) and homologous T3 (DSPAα1 seeds, around 50 mg) was extracted using a P-PER® Plant Protein Extraction Kit (Thermo Scientific, Waltham, USA). His-tagged protein was purified with Ni-NTA by gravity-flow chromatography (Qiagen, Venlo, Netherlands). 1 ml Ni-NTA slurry (0.5 ml bed volume) was transferred via pipette to a 1.7-ml microcentrifuge tube and centrifuge at 500×g for 5 min at 4° C. The supernatant was removed, and 1 ml of Buffer A [50 mM NaH₂PO₄, 300 mM NaCl, pH8.0] was added. The slurry was mixed by gentle inversion. The centrifugation step at 500×g was repeated for 5 min at 4° C. and the supernatant was removed. The slurry was then ready to mix with the isolated total protein solution (described above). The total protein extract was added to this equilibrated Ni-NTA slurry and shaken with a rocker (Boekel Scientific, Feasterville, USA) for 1 hour at 4° C. After 1 hour, the protein-extract/Ni-NTA mixture was transferred into a Polypropylene Column (Cat. No. 34924, Qiagen) equilibrated with Buffer B [50 mM NaH₂PO₄, 300 mM NaCl, 5 mM imidazole, pH8.0]. The column was then washed with 10 bed volumes (5-ml) of Buffer B. The bound His-tagged protein was then eluted with 200 μl Buffer C [50 mM NaH₂PO₄, 300 mM NaCl, 1M imidazole, pH8.0] twice into separate tubes. The resulting elutants were used for protein concentration measurement, the fibrin plate assay and the blood clot dissolving test.

Fibrin Plate Assay

Fibrinolytic enzyme activity was detected by a modified fibrin plate method (Li et al 2012). 50 mL of 0.5% agarose in 1×PBS buffer was boiled in a 200 mL conical flask and left to cool in a 40° C. water bath. 1 mg/mL of fibrinogen, 0.1 IU/mL of thrombin, and 0.1 IU/mL plasminogen were added and swirled to mix. The mixture was slowly poured into the petri dish and the plate was left undisturbed until the agarose solidified. Wells (3 mm diameter) were formed in each plate with an aseptic hole punch. 50 μL of elutant samples (0.5 mg protein/mL) were loaded into each well and the plates were incubated at room temperature overnight.

Blood-Clot Lysis Activity Assay

An in vitro human blood-clot lysis activity assay was used as described by Li et al (2012). Whole blood was received from Sanguine Biosciences, Inc. (Valencia, Calif., USA). Approximately 50 mg blood clots were isolated and rinsed with 1×PBS and placed in the wells of a 24-well plate. 50 μL of protein elutant from seeds was mixed in 450 μL of 1×PBS buffer and added to the wells of the 24-well plate (Greiner Bio-One, Monroe, USA) containing clots. Treated samples were incubated at 37° C. overnight.

Results and Conclusions:

cDNAs encoding the full length wild-type t-PA, codon optimized t-PA and vampire bat DSPAα1 and DSPAα2 proteins were cloned into a plant vector system. Generally, the full length genes were redesigned to preferentially match the codon frequencies of the host tobacco plant without altering the amino acid sequence of the proteins. In order to increase recombinant protein yields in plant cell compartments, the native signal peptides were replaced with the plant optimized murine mAb24 heavy chain (LPH). These targeting sequences enabled transport of the proteins to the apoplast and vacuole in different secretory pathways. All gene sequences were flanked by C-terminal 6×His tags for protein purification and included a KDEL (SEQ ID NO: 14) sequence for retention of recombinant protein in the endoplasmic reticulum (ER).

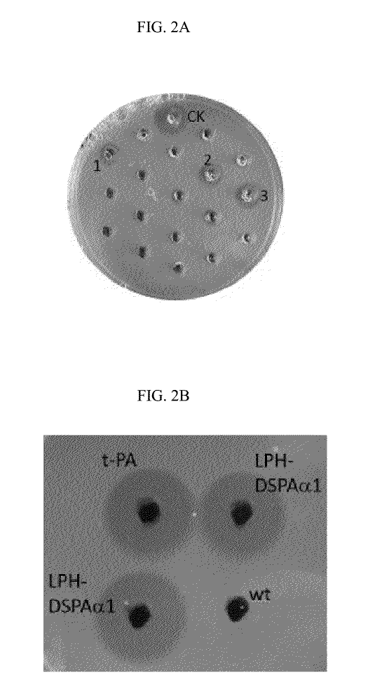

The His-tagged proteins were purified from total soluble protein from immature seeds by nickel-chelating affinity chromatography. The functional t-PA and DSPA proteins were screened by a fibrin degradation assay. The results showed that recombinant t-PA from T1 seeds and DSPAα1 from T3 homologous seeds can degrade fibrin, as shown in FIGS. 2A and B. Purified t-PA and DSPAα1 protein showed a half-transparent lytic area on the fibrin plate, indicating that fibrin had been degraded into soluble peptides. The recombinant proteins were able to degrade fibrin even after 24 hours at room temperature, indicating that the fibrin degradation activity of proteins isolated from dry seeds is very robust. In contrast, protein eluant from non-transgenic wild seeds displayed no fibrin cleaving activity.

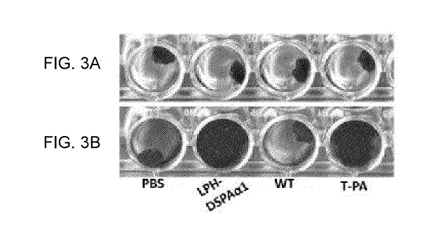

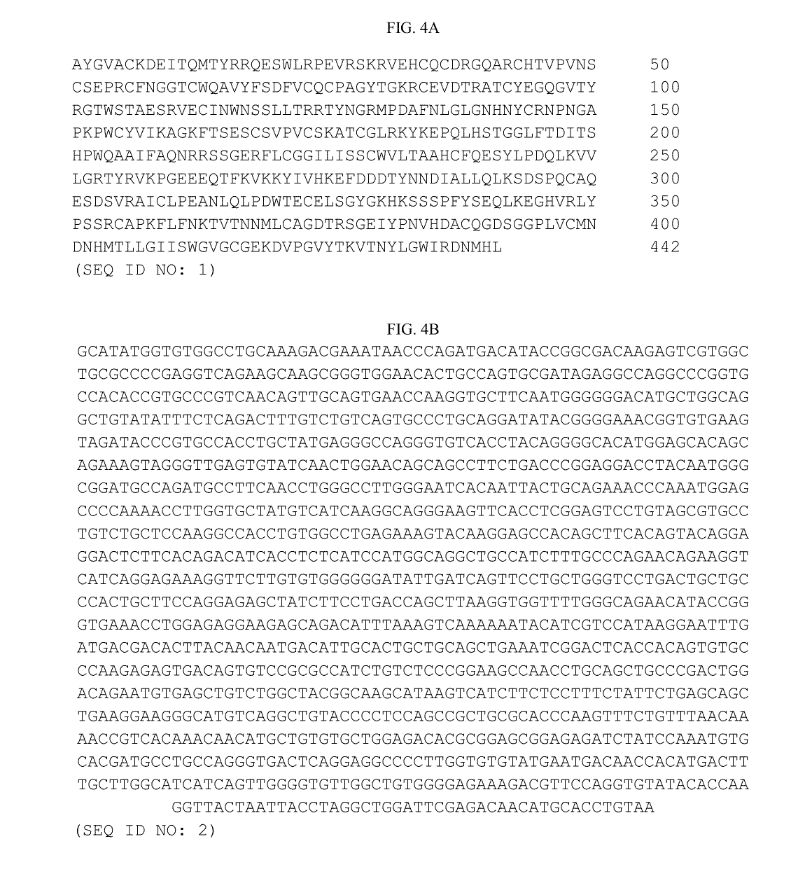

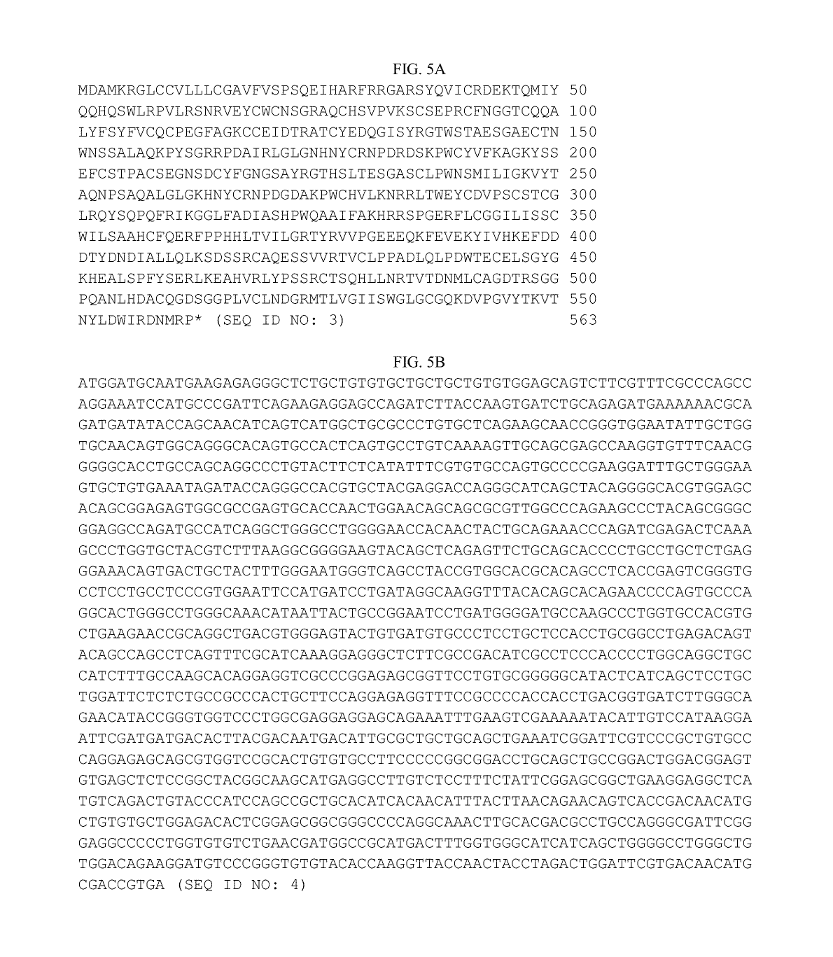

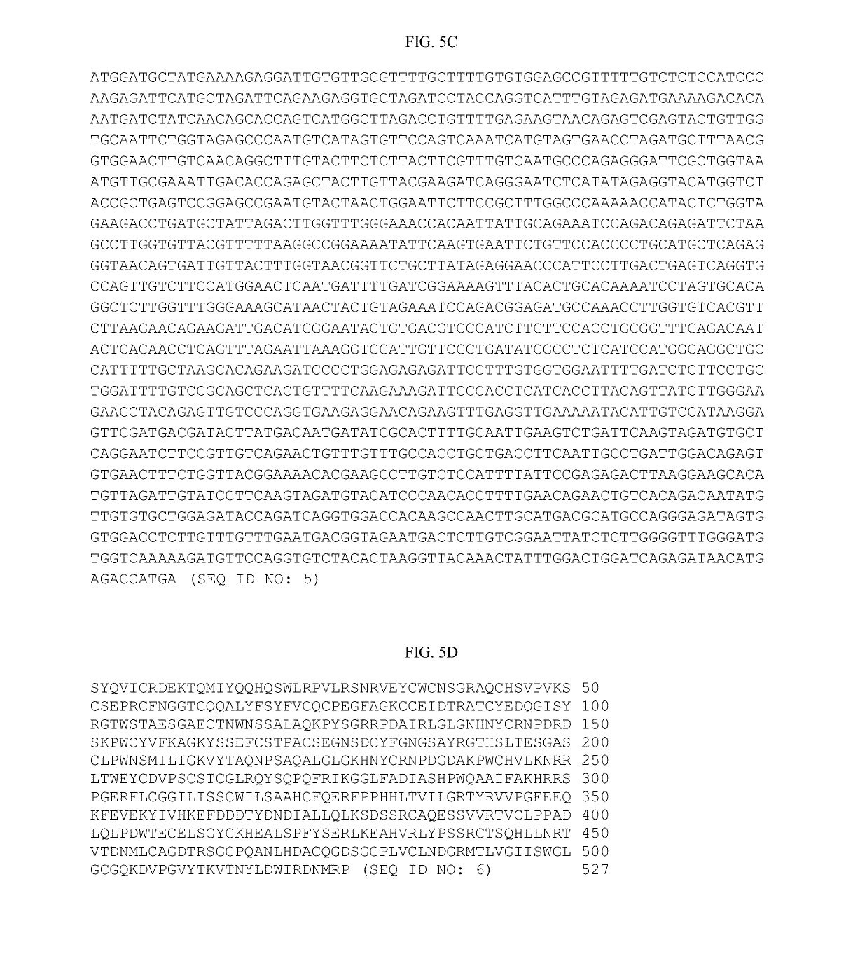

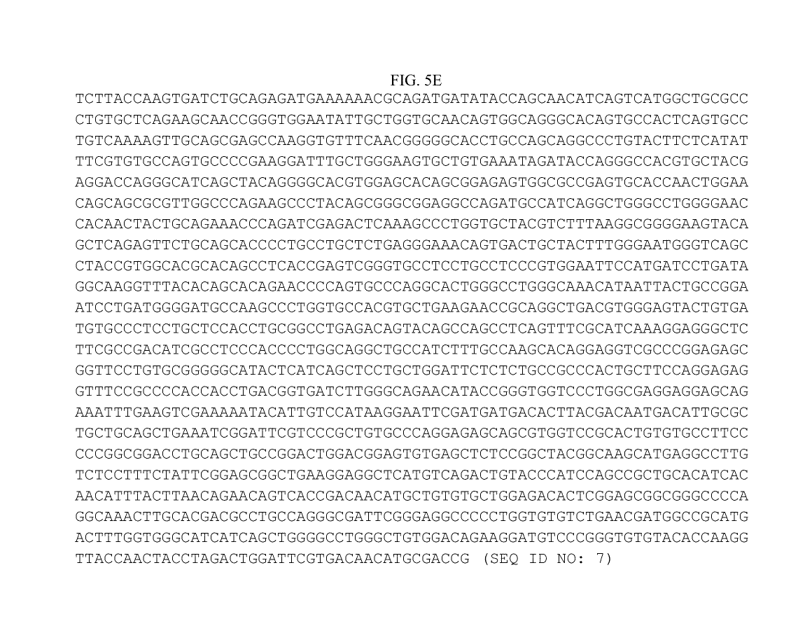

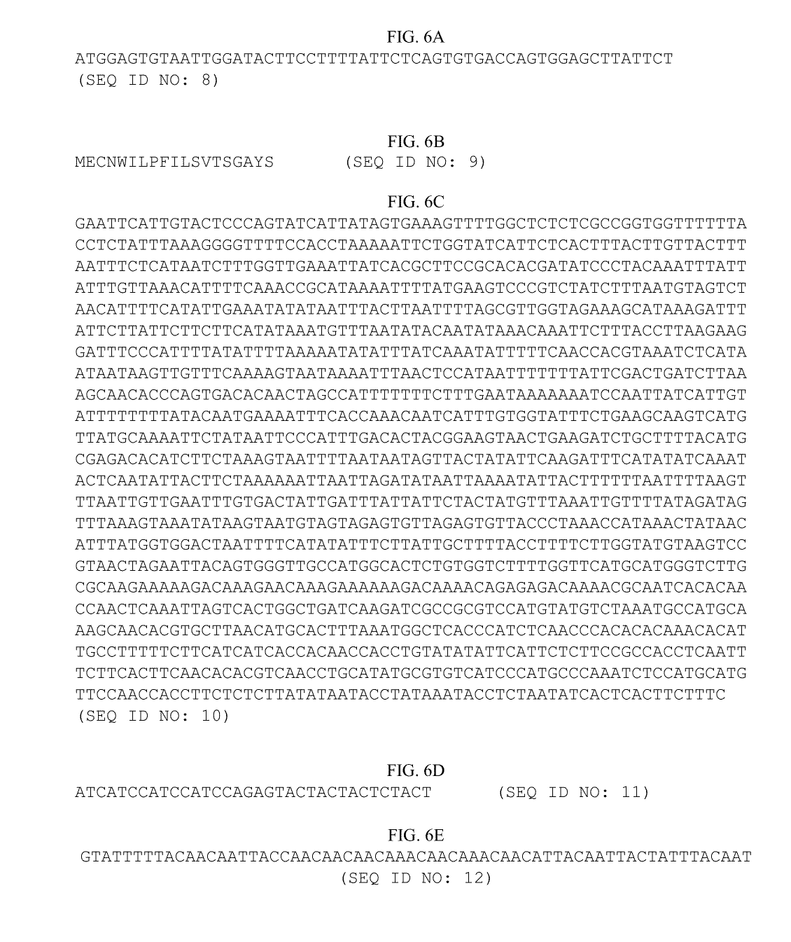

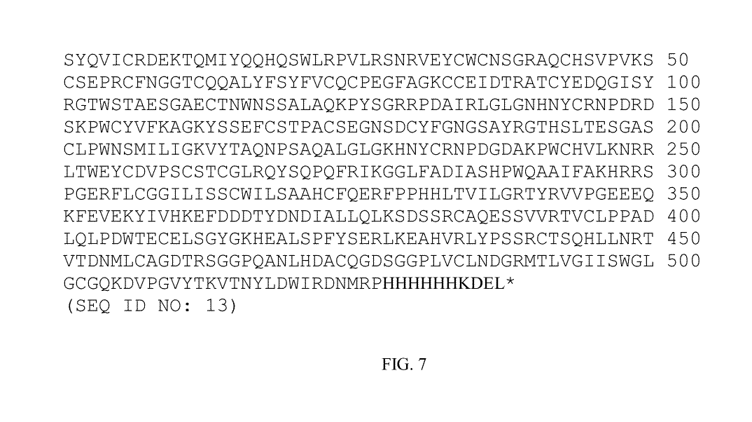

Significantly, testing showed that the DSPAα1 produced in transgenic seeds significantly dissolved blood clots (FIG. 3). No evidence of clot lysis was observed when blood samples were treated with the non-transgenic wild tobacco seeds. Similar results were obtained with t-PA recombinant protein (e.g. see FIG. 3). These findings indicate that plant seed systems are excellent platforms for production of functional blood clot dissolving proteins.

In conclusion, by using a seed-specific promoter, transgenic tobacco plants have been generated in which t-PA, DSPAα1 and DSPAα2 production is targeted to seeds. The data showed that recombinant proteins t-PA, DSPAα1 and DSPAα2 produced in this manner can degrade fibrin and DSPAα1 significantly dissolves human blood clots. Thus, transgenic plants can be used to produce active, safe, and inexpensive therapeutic proteins. In particular, plant seed-based platforms can be used for large scale and low cost production of functional proteins that dissolve blood clots.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

### REFERENCES

Adams H P, del Zoppo G, Alberts M J, Bhatt D L, Brass L and et al (2007) Guidelines for the early management of adults with ischemic stroke. Stroke 38:1655-1711.
Bock R and Warzecha H (2010) Solar-powered factories for new vaccines and antibiotics. Trends Biotechnol 28:246-252.
Bringmann P, Gruber D, Liese A, Toschi L, Kratzchmar J, Schleuning W D and Donner P (1995) Structural features mediating fibrin selectivity of vampire bat plasminogen activators. J Biol Chem 270:25596-25603.
Dafer R M and Biller J (2007) Desmoteplase in the treatment of acute ischemic stroke. Expert Rev Neurother. 7:333-337.
Dorana P M (2006) Foreign protein degradation and instability in plants and plant tissue cultures. Trends Biotechnol. 24: 426-432.
Furlan A J, Eyding D, Albers G W, Al-Rawi Y, Lees K L et al (2006). Dose Escalation of desmoteplase for acute ischemic stroke (DEDAS): evidence of safety and efficacy 3 to 9 hours after stroke onset. Stroke 37:1227-1231.
Grandjean C, McMullen P C and Newschwander G (2004) Vampire bats yield potent clot buster for ischemic stroke. J Cardiovasc Nurs 19:417-420.
Furlan A J, Eyding D, Albers G W, Al-Rawi Y, Lees K L et al (2006). Dose Escalation of desmoteplase for acute ischemic stroke (DEDAS): evidence of safety and efficacy 3 to 9 hours after stroke onset. Stroke 37:1227-1231.
Hacke W, Kaste M, Bluhmki E, Brozman M, Davalos A and et al (2008). Thrombolysis with alteplase 3 to 4.5 hours after acute ischemic stroke. N Engl J Med. 359:1317-1329.
Kratzschmar J, Haendler B, Langer G, Boidol W, Bringmann P, et al (1991) The plasminogen activator family from the salivary gland of the vampire bat Desmodus rotundus: cloning and expression. Gene 105:229-237.
Krätzschmar J, Haendler B, Bringmann P, Dinter H, Hess F, Donner P and Schleuning W D (1992) High-level secretion of the four salivary plasminogen activators from the vampire bat Desmodus rotundus by stably transfected baby hamster kidney cells. Gene 116:281-284.
Li G, Wang K Y, Li D, Wang N, and Liu D (2012) Cloning, expression and characterization of a gene from earthworm Eisenia fetida encoding a blood-clot dissolving protein.
PLoS One. 2012; 7(12):e53110. doi: 10.1371/journal.pone.0053110.
Lijnen H R and Collen D (1987) Tissue-type plasminogen activator. Ann Biol Clin (Paris). 45:198-201.
Lijnen H R and Collen D (2000) Molecular basis of thrombolytic therapy. J. Nucl. Cardiol. 7:373-81.
Lilie H, Schwarz E and Rudolph R (1998) Advances in refolding of proteins produced in E. coli. Curr Opin Biotechnol 9:497-501.
Ma J K, Barros E, Bock R, Christou P, Dale P J, Dix P J, Fischer R, Irwin J, et al (2005) Molecular farming for new drugs and vaccines: Current perspectives on the production of pharmaceuticals in transgenic plants. EMBO Rep 6:593-599.
Nuttall J, Vine N, Hadlington J L, Drake P, Frigerio L, and Ma J K (2002) E R-resident chaperone interactions with recombinant antibodies in transgenic plants. Eur. J. Biochem. 269:6042-6051.
Peterson R K D and Arntzen C J (2004) On risk and plant-based biopharmaceuticals. Trends Biotechnol 22:64-66.
Spök A, Twyman R M, Fischer R, Ma J and Sparrow P (2008) Evolution of a regulatory framework for pharmaceuticals derived from genetically modified plants. Trends Biotechnol 26 (9): 506-517.
Suzuki Y, Nagai N, Yamakawa K, Kawakami J, Lijnen H R and Umemura K (2009). Tissue-type plasminogen activator (t-PA) induces stromelysin-1 (MMP-3) in endothelial cells through activation of lipoprotein receptor-related protein. Blood 114:3352-3358.
Tsirka S E, Gualandris A, Amaral D G and Strickland S (1995) Excitotoxin-induced neuronal degeneration and seizure are mediated by tissue plasminogen activator. Nature 377:340-344.

What is claimed is:

1. A transgenic seed comprising a protein that dissolves blood clots.

2. The transgenic seed of claim 1, wherein the transgenic seed is from a plant type selected from the group consisting of tobacco, rice, maize and soybean.

3. The transgenic seed of claim 1, wherein the protein that dissolves blood clots is Desmodus rotundus salivary plasminogen activator (DSPA) or human tissue plasminogen activator (t-PA).

4. The transgenic seed of claim 3, wherein the DSPA is or includes an amino acid sequence as set forth in SEQ ID NO: 1 and the t-PA is or includes an amino acid sequence as set forth in SEQ ID NO: 6.

5. A transgenic plant or progeny thereof, comprising:
a nucleic acid sequence comprising a nucleotide sequence encoding a protein that dissolves blood clots operably linked to a seed specific or selective promoter.

6. The transgenic plant or progeny thereof of claim 5, wherein the transgenic plant or progeny thereof is a type of plant selected from the group consisting of tobacco, rice, maize and soybean.

7. The transgenic plant or progeny thereof of claim 5, wherein the protein that dissolves blood clots is Desmodus rotundus salivary plasminogen activator (DSPA) or human tissue plasminogen activator (t-PA).

8. The transgenic plant or progeny thereof of claim 5, wherein the seed specific or selective promoter is a phaseolin promoter or a napin promoter.

9. A method of making a recombinant protein that dissolves blood clots, comprising:
genetically engineering a plant cell or a plant explant to contain and express a nucleotide sequence encoding a protein that dissolves blood clots operably linked to a seed specific or selective promoter;
cultivating the plant cells or plant explant so as to produce a transgenic plant,
cultivating the transgenic plant so as to produce seeds comprising the protein that dissolves blood clots;
harvesting the seeds; and
isolating the protein that dissolves blood clots from the seeds.

10. A vector comprising a nucleotide sequence encoding a protein that dissolves blood clots operably linked to a seed specific or selective promoter.

11. The vector of claim 10, wherein the nucleic acid sequence includes a nucleotide sequence as set forth in SEQ ID NO 2, SEQ ID NO: 4 or SEQ ID NO: 5.

12. The vector of claim 10, wherein the nucleic acid sequence encoding a protein encodes an amino acid sequence which is or includes an amino acid sequence as set forth in SEQ ID NO: 1, or an amino acid sequence which is or includes an amino acid sequence as set forth in SEQ ID NO: 6.

13. A nucleotide sequence comprising
a nucleotide sequence encoding a blood clot-dissolving protein operably linked to a seed specific or selective promoter.

14. The nucleotide sequence of claim 13, wherein the blood clot-dissolving protein is DSPA.

15. The nucleotide sequence of claim 14, wherein the DSPA is or includes an amino acid sequence as set forth in SEQ ID NO: 1.

16. The nucleotide sequence of claim 14, wherein the DSPA is encoded by a nucleotide sequence that is or includes a nucleotide sequence as set forth in SEQ ID NO: 2.

17. The nucleotide sequence of claim 13, wherein the blood clot-dissolving protein is t-PA.

18. The nucleotide sequence of claim 17, wherein the t-PA is or includes an amino acid sequence as set forth in SEQ ID NO: 6.

19. The nucleotide sequence of claim 17, wherein the t-PA is encoded by a nucleotide sequence that is or includes a nucleotide sequence as set forth in SEQ ID NO: 4 or SEQ ID NO: 5.

20. The nucleotide sequence of claim 13, wherein the seed specific or selective promoter is or includes a nucleotide sequence as set forth in SEQ ID NO: 10.

21. A recombinant protein comprising an amino acid sequence as set forth in SEQ ID NO: 1 or SEQ ID NO: 6.
